(19) United States
(12) Patent Application Publication
(10) Pub. No.: US 2026/0108753 A1
(43) Pub. Date: Apr. 23, 2026

(54) Ergonomic Applicator Head for Skincare

(71) Applicant: SHENZHEN NUON MEDICAL EQUIPMENT CO., LTD., Shenzhen (CN)

(72) Inventors: Alain Dijkstra, Amstelveen (NL); LI XIANG, Shenzhen (CN); Tang Yuanzhou, Shenzhen (CN); Huang Jianlong, Shenzhen (CN)

(73) Assignee: SHENZHEN NUON MEDICAL EQUIPMENT CO., LTD., Shenzhen (CN)

(21) Appl. No.: 19/325,492

(22) Filed: Sep. 11, 2025

(30) Foreign Application Priority Data
Oct. 17, 2024 (CN) 2024225146035

(51) Int. Cl. A61N 5/06; A45D 40/26

(52) U.S. Cl. CPC A61N 5/0616; A45D 40/261; A45D 2200/205; A61N 2005/0626; A61N 2005/0632; A61N 2005/0644; A61N 2005/0651; A61N 2005/0659; A61N 2005/0663

(57) ABSTRACT

An applicator head comprising a main body includes a housing and a connecting portion configured to detachably connect with a cosmetic container. A first side of the housing has at least one phototherapy element and at least one rolling element. A second side of the housing includes a display configured to provide visual information. A fluid channel is in fluid communication with the cosmetic container and the first side of the housing. The first and second sides of the housing are arranged at an inclined orientation relative to a central axis of the main body for ergonomic application. The applicator head further includes stimulation elements and a skin detection sensor configured to detect skin parameters, adjust treatment parameters based on real-time skin data, and provide feedback.

### TECHNICAL FIELD

The present invention relates to the technical field of skincare devices and personal care product containers. More particularly, it pertains to an applicator head having multifunctional benefits. Specifically, the invention provides an applicator head configured for the application of cosmetic or therapeutic fluids to the skin, incorporating features such as phototherapy, mechanical stimulation, skin parameter sensing, and real-time feedback through a user interface.

### BACKGROUND

In recent years, the demand for home-use skincare and cosmetic treatment devices has significantly increased. Consumers seek convenient, efficient, and personalized skincare fluids that can replicate or supplement professional treatments. As a result, various skincare applicators have been developed to assist in the application and absorption of cosmetic formulations.

Currently, many conventional skincare devices are limited in functionality. A common type of device typically connects to the mouth of a skincare fluid container at one end, while the opposite end includes an applicator interface for dispensing the product onto the user's skin. These devices often rely solely on passive application, such as simple rolling or wiping, and lack integrated features to enhance absorption or adapt treatment to individual skin conditions.

Additionally, most existing devices do not provide real-time monitoring of skin parameters, such as hydration level, temperature, oil content, PH level, or SPF. Without this feedback, the treatment process is generic and cannot be tailored to the user's current skin needs. Furthermore, users often cannot track treatment effectiveness or receive guidance on optimal use, which can reduce efficacy and user satisfaction.

Another challenge with conventional designs is ergonomic inefficiency. The straight-line configuration of most applicators makes it difficult for users to maintain clear visual feedback (e.g., from a display screen) while applying treatment, especially when using phototherapy or stimulation modes. The lack of ergonomic design also limits the precision and comfort of the application.

There is therefore a need for a multifunctional skincare applicator head that not only enables effective dispensing of cosmetic fluids but also integrates advanced treatment modalities such as phototherapy, micro-stimulation, and massage, provides real-time monitoring of skin conditions/parameters, and offers responsive feedback to guide and optimize treatment. Furthermore, there is a need for an applicator with an ergonomic and inclined structure that allows simultaneous treatment and visual interaction with a user interface.

The present invention addresses these limitations by providing an improved applicator head configured for detachable connection with a cosmetic container, and incorporating features such as stimulation elements, rolling massage, skin detection sensors, a control unit, and a display unit, all integrated into an ergonomically inclined housing structure. This enables enhanced treatment delivery, responsive personalization, and user convenience.

### OBJECTS OF THE INVENTION

An object of the present invention is to provide a multifunctional applicator head that can be detachably connected to a cosmetic container for the effective application of cosmetic or skincare fluids.

Another object of the invention is to provide an applicator head that integrates stimulation components and rolling massage elements to enhance skin absorption and treatment efficacy.

A further object of the invention is to provide an applicator head equipped with at least one skin detection sensor for detecting skin parameters such as hydration, temperature, oil content, SPF, or impedance in real time.

Yet another object of the invention is to enable the dynamic adjustment of treatment parameters (e.g., stimulation intensity, light wavelength, fluid dispensing rate) based on the detected skin conditions, thereby allowing for personalized skincare treatment.

It is also an object of the invention to provide a user interface, such as a display screen, capable of providing visual feedback, treatment information, or user interaction during the application.

An additional object of the invention is to offer an ergonomic, inclined housing design that positions the applicator's treatment surface and display at an angle to improve user comfort, precision, and visibility during use.

Another object of the invention is to incorporate wireless communication and data storage capabilities to enable treatment tracking, mobile device connectivity, and enhanced user experience.

### SUMMARY OF THE INVENTION

The present invention relates generally to cosmetic devices for delivering cosmetic treatments to human skin, and more particularly to an applicator head comprising a housing configured for integration with a cosmetic container and capable of combining targeted product dispensing with skin stimulation technologies such as phototherapy, electrotherapy, magneto therapy, micro-current therapy, massage, temperature control, and sensor-based feedback. The invention also relates to a method for delivering cosmetic treatments using such an applicator head.

To achieve this objective, the present invention provides an applicator head comprising a main body that includes a housing and a connecting portion coupled to the housing. The connecting portion is configured to detachably connect with a cosmetic container. A first side of the housing includes at least one stimulation element and at least one rolling element. A second side of the housing is equipped with a display and at least one skin detection sensor configured to provide visual information. A fluid channel is in fluid communication with both the cosmetic container and the first side of the housing. The first side and the second side of the housing are arranged at an inclined orientation relative to a central axis of the main body. The inclined orientation enhances user comfort, precision of application, and visibility during use through an ergonomic design.

In one embodiment of the invention, at least one stimulation element is selected from the group consisting of a phototherapy component, electrotherapy component, magnotherapy component, micro-current component, heating element, cooling element, Peltier element, vibrational element for massage, ultrasonic wave component, or other forms of therapeutic components. In one embodiment of the invention, the housing is cylindrical. However, in other embodiments, the housing may be non-cylindrical in shape.

In one embodiment of the invention, the display includes a touchscreen display for real-time monitoring of therapy settings and skin parameter data.

In one embodiment of the invention, the connecting portion comprises a threaded connector, bayonet mount, snap-fit mechanism, or magnetic locking mechanism to detachably connect with the cosmetic container.

In one embodiment of the invention, the first side of the housing includes a mounting member, at least one stimulation element, and at least one rolling element. The mounting member is located in the opening on the first side of the housing and is connected to the housing. The rolling element is mounted on a side of the mounting member facing away from the second side of the housing. The mounting member has a raised portion on a side facing the second side of the housing. A second fluid channel is disposed on the mounting member and the raised portion. The second fluid channel extends from a side of the raised portion proximal to the connecting portion to a side of the mounting member facing away from the second side of the housing. The first fluid channel and the second fluid channel are connected.

In one embodiment of the invention, the rolling element includes at least one ball. The mounting member is provided with at least one receiving groove on a side facing away from the second side of the housing. The receiving groove is provided in a one-to-one correspondence with the ball, and the ball is movably mounted within the corresponding receiving groove. The second fluid channel is connected to at least one of the receiving grooves.

In one embodiment of the invention, the second side of the housing includes a display screen and a cover. The cover covers an opening on the second side of the housing and is connected to the housing. The cover is formed with a mounting groove, the opening of which faces away from the first side of the housing, and the display screen is mounted within the mounting groove.

In one embodiment of the invention, the cover is provided with heat dissipation holes extending through the cover. The heat dissipation holes are spaced apart from the mounting grooves in the radial direction of the housing.

In one embodiment of the invention, the applicator head includes a cooling fan, which is disposed within the inner hole of the housing and between the first side of the housing and the cover. The cooling fan is configured to drive air within the inner hole of the housing from the side proximate to the first side of the housing to the side proximate to the cover.

In one embodiment of the invention, the mounting slot includes a bottom wall positioned opposite its opening, the bottom wall being provided with multiple ribs, and the bottom wall and any two adjacent ribs forming a heat dissipation channel; and/or, the mounting slot includes a bottom wall positioned opposite its opening and side walls surrounding the bottom wall, at least one of the bottom wall and the side walls being provided with ventilation holes, the mounting slot communicating with the inner hole of the housing or the heat dissipation holes through the ventilation holes.

In one embodiment of the invention, the applicator head further includes a skin detection sensor and a control unit. The skin detection sensor is mounted on the housing, which may be located on the first side, the second side, or the connecting portion. The control unit is mounted within the housing and is signal-connected to the skin detection sensor and the second side of the housing, respectively.

The skin detection sensor is configured to detect skin condition and send the feedback to the control unit. The control unit is configured to control the second side of the housing to display the detection result fed back by the skin detection sensor.

In one embodiment of the invention, the skin detection sensor is selected from the group consisting of a temperature sensor, a moisture sensor, an impedance sensor, an optical sensor, a SPF sensor/UV camera, or a combination thereof.

In one embodiment of the invention, the skin detection sensor is configured to detect at least one of the water level, oil level, temperature, SPF, and hydration levels of the user's skin.

In one embodiment of the invention, the control unit is programmed with multiple treatment modes, including pre-set therapy cycles based on skin conditions.

In one embodiment of the invention, the control unit is configured to store user treatment history and provide customized therapy recommendations.

In one embodiment of the invention, the applicator head further includes a mounting bracket and a circuit component. The mounting bracket is mounted in the housing, and the circuit component is mounted on the mounting bracket. The second side of the housing and the first side of the housing are electrically connected to the circuit component.

In one embodiment of the invention, the applicator head further comprises a rechargeable power source electrically connected to the circuit assembly.

In one embodiment of the invention, the circuit assembly includes a wireless communication module for connectivity with external devices.

In one embodiment of the invention, the applicator head further comprises a mobile application that allows a user to control the applicator head remotely via Bluetooth or Wi-Fi connectivity.

In one embodiment of the invention, the applicator head host features an automatic shut-off function after a predefined therapy duration to prevent overuse.

In one embodiment of the invention, the first side of the housing includes at least one positive electrode terminal and at least one negative electrode terminal, each of which is electrically connected to the circuit component.

The first side of the housing at least one light-emitting diode, which is electrically connected to the circuit component. The applicator head further includes a control unit, and the first side of the housing includes a semiconductor cooling element, which is electrically connected to the circuit component via the control unit. The control unit is configured to switch the direction of power flow to the semiconductor cooling element.

The applicator head provided by this application has advantages compared to the prior art. By mounting the multiple elements like phototherapy element, rolling element, stimulation element etc. on the first side of the housing and positioning the skin detection sensor and the display screen on the second side of the housing, with the first side of the housing and the second side of the housing positioned at an inclined orientation relative to a central axis of the main body can appropriately increase the dimensions of the housing, resulting in a larger outer contour of the two sides of the housing.

This increases the space available for mounting the multiple elements within the applicator head. This, in turn, increases the contact area with the user's skin, improving the delivery of the cosmetic fluid (which may be an essence, lotion, or water, etc.) to the skin.

The second side of the housing can be used to display information such as the head's operating status, skin conditions, SPF, remaining battery power, or usage time, thereby enriching the device's functionality.

In the context of this specification, terms like “light”, “radiation”, “irradiation”, “emission” and “illumination”, etc. refer to electromagnetic radiation in frequency ranges varying from the visible frequencies to Infrared (IR) frequencies and wavelengths, wherein the range is inclusive of visible light, IR frequencies and wavelengths. Preferably, it refers to low-level electromagnetic radiation of low-level red and near-infrared (NIR) light. It is to be noted here that IR radiation can be categorized into several categories according to respective wavelength ranges, which are again envisaged to be within the scope of this invention. A commonly used subdivision scheme for IR radiation includes Near IR (0.75-1.4 μm), Short-Wavelength IR (1.4-3 μm), Mid-Wavelength IR (3-8 μm), Long-Wavelength IR (8-15 μm), and Far IR (15-1000 μm). In this regard, light application is at relatively low energy densities, typically below about 500 mW, as compared to other forms of laser therapy that are used for ablation, cutting, and thermally coagulating tissue.

In the context of the specification, the term “light source” or “phototherapy source” etc. refers to a source emitting coherent laser light, or light-emitting diodes (“LEDs”). The term “light therapy” refers to light generated from any of the sources, such as lasers, LED sources, or super luminous diodes (“SLD”).

In the context of the specification, when an element is referred to as being “fixed to” or “disposed to” another element, it may either be directly on another element or indirectly on that other element. When a component is said to be “connected” or “connected to” another component, it may be directly connected to another component or indirectly connected to other components on the piece.

In the context of the specification, the terms “first”, “second” and “third” are only used for descriptive purposes and do not implicate the relative importance or implicitly indicate the quantity of technical features indicated.

In the context of the specification, “Light Emitting Diodes (LEDs)” refer to semiconductor diodes capable of emitting electromagnetic radiation when supplied with an electric current. The LEDs are characterized by superior power efficiencies, smaller sizes, rapid switching speeds, physical robustness, and longer lifespans compared to incandescent or fluorescent lamps. The one or more LEDs may include through-hole type LEDs (generally emitting electromagnetic radiation in red, green, yellow, blue, and white colors), Surface Mount Technology (SMT) LEDs, Bi-color LEDs, Pulse Width Modulated RGB (Red-Green-Blue) LEDs, and high-power LEDs, among others.

Materials used in one or more LEDs may vary from one embodiment to another, depending upon the frequency of radiation required. Different frequencies can be obtained from LEDs made from pure or doped semiconductor materials. Commonly used semiconductor materials include nitrides of Silicon, Gallium, Aluminum, Boron, Zinc Selenide, etc., in pure form or doped with elements such as Aluminum and Indium etc. For example, red and amber colors are produced from Aluminum Indium Gallium Phosphide (AlGaInP) based compositions, while blue, green, and cyan use Indium Gallium Nitride based compositions. White light may be produced by mixing red, green, and blue lights in equal proportions, while varying proportions may be used to generate a wider color gamut. White and other colored lightings may also be produced using phosphor coatings such as Yttrium Aluminum Garnet (YAG) in combination with a blue LED to generate white light, and Magnesium-doped potassium fluorosilicate in combination with a blue LED to generate red light.

In some embodiments, the display includes an SPF mirror (or the camera behind it) that operates on the principle of ultraviolet (UV) light absorption by sunscreen formulations. When sunscreen is applied, the UV-blocking ingredients absorb or scatter the UV radiation, causing the covered areas of the skin to appear significantly darker in the mirror or on the display, while unprotected or unevenly covered areas appear lighter. This contrast enables users to observe the distribution, thickness, and uniformity of sunscreen application, thereby highlighting gaps or insufficient coverage that are not visible under normal lighting conditions.

The present invention provides a display configured to indicate the measured or calculated SPF (Sun Protection Factor) value, and a surrounding arrangement of ultraviolet (UV) light-emitting diodes (LEDs) disposed circumferentially around the display. The UV LEDs are adapted to irradiate a target area, such as skin or a substrate coated with sunscreen, thereby enabling visual or sensor-based observation of the sunscreen's protective efficacy against UV radiation. The display at the center operates in conjunction with the UV illumination to present the SPF value in a clear and user-friendly manner, offering a compact, integrated system for evaluating sunscreen performance in real time.

In the context of the specification, the term “applicator head” broadly includes any handheld or modular unit capable of delivering one or more therapeutic functions.

In the context of the specification, the term “Stimulation elements” may include, but are not limited to, electrotherapy, micro-current, magneto therapy, cooling, heating, vibration, electrical pulses, or other forms of therapeutic output.

### BRIEF DESCRIPTION OF THE ACCOMPANYING DRAWINGS

To more clearly illustrate the technical fluids in the embodiments of this application, the following briefly introduces the drawings required for use in the embodiments or prior art descriptions. Obviously, the drawings described below represent only some embodiments of this application. Persons skilled in the art can derive other drawings based on these drawings without inventive effort.

FIG. 1 is a schematic structural diagram of the applicator head, provided in one embodiment of this application.

FIG. 2 is an exploded view of the applicator head, provided in one embodiment of this application.

FIG. 3 is a schematic structural diagram of the applicator head, provided in one embodiment of this application, from another perspective.

FIG. 4 is a schematic cross-sectional view of the applicator head in FIG. 3 taken along the D-D direction.

FIG. 5 is a schematic structural diagram of the mounting components of the first side of the housing, provided in one embodiment of this application.

FIG. 6 is a schematic structural diagram of the cover, provided in one embodiment of this application.

### DETAILED DESCRIPTION

Embodiments of the present invention disclosure will be described more fully hereinafter with reference to the accompanying drawings in which like numerals represent like elements throughout the fugures, and in which example embodiments are shown.

The detailed description and the accompanying drawings illustrate the specific exemplary embodiments by which the disclosure may be practiced. These embodiments are described in detail to enable those skilled in the art to practice the invention illustrated in the disclosure. It is to be understood that other embodiments may be utilized, and other changes may be made, without departing from the spirit or scope of the present disclosure. The following detailed description is therefore not to be taken in a limiting sense, and the scope of the present invention disclosure is defined by the appended claims. Embodiments of the claims may, however, be embodied in many different forms and should not be construed as limited to the embodiments set forth herein.

It should be noted that when an element is referred to as being “fixed to” or “disposed on” another element, it may be directly or indirectly attached to the other element. When an element is referred to as being “connected to” another element, it may be directly or indirectly connected to the other element.

It should be understood that the terms “length,” “width,” “upper,” “lower,” “front,” “back,” “left,” “right,” “vertical,” “horizontal,” “top,” “bottom,” “inner,” “outer,” etc., indicating positions or relationships, are based on the positions or relationships shown in the accompanying drawings and are intended solely for the purpose of facilitating the description of this application and simplifying the description. They do not indicate or imply that the structures or elements referred to must have, be constructed, or operate in a specific orientation. Therefore, they should not be construed as limiting this application.

Furthermore, the terms “first” and “second” are used for descriptive purposes only and should not be construed as indicating or implying relative importance or implicitly specifying the number of technical features indicated. Therefore, features specified as “first” or “second” may explicitly or implicitly include one or more of such features. In the description of this application, “plurality” means two or more, unless otherwise specifically defined. To illustrate the technical fluids described in this application, the following detailed description is provided with reference to specific figures and embodiments.

Embodiments of the present invention disclose an applicator head comprising a main body that includes a housing and a connecting portion configured to detachably connect with a cosmetic container. A first side of the housing has at least one stimulation element and at least one rolling element. A second side of the housing includes a display configured to provide visual information and a skin detection sensor. A fluid channel is in fluid communication with the cosmetic container and the first side of the housing. The first and second sides of the housing are arranged at an inclined orientation relative to a central axis of the main body for ergonomic application. The applicator head further includes a skin detection sensor configured to detect skin conditions/parameters, adjust treatment parameters based on real-time skin data, and provide feedback.

The stimulation element comprises multiple light-emitting diodes (LEDs) configured to emit light in a wavelength range selected from red, blue, green, purple, or near-infrared as required for targeted skincare treatments. The stimulation element is selected from the group consisting of a phototherapy component, electrotherapy component, magnotherapy component, micro-current component, heating element, cooling element, Peltier element, vibrational element for massage, ultrasonic wave component, or other forms of therapeutic components. A rechargeable battery or power source configured to supply power to the phototherapy element and display.

The rolling element is configured to rotate freely to provide a massage effect during application of the cosmetic fluid to the skin. The rolling element is made of a material selected from stainless steel, jade stone, ceramic, or a thermally conductive polymer to provide cooling or heating during application.

The display is a touchscreen configured to provide real-time monitoring of both therapy parameters (such as duration, mode, and intensity) and skin parameter readings (such as hydration, SPF, or temperature levels). The display may include an SPF mirror to highlight gaps or insufficient coverage of sunscreen that is not visible under normal lighting conditions.

The skin detection sensor is selected from the group consisting of a temperature sensor, a moisture sensor, an impedance sensor, an optical sensor, a SPF sensor/UV camera, or a combination thereof. The skin detection sensor is configured to detect at least one of the water level, oil level, temperature, SPF, or hydration level of the skin. The control unit includes a wireless communication module for connectivity for connection to a mobile device to transmit skin data and receive treatment settings. The control unit is programmed with multiple treatment modes, including pre-set therapy cycles based on skin conditions/parameters. The control unit is configured to store user treatment history and provide customized therapy recommendations.

The first side of the housing and the second side of the housing are electrically connected to a circuit component. The first side of the housing includes at least one positive electrode terminal and at least one negative electrode terminal, each of which is electrically connected to the circuit component.

A method is provided for delivering a cosmetic treatment to a user's skin using an applicator head. The method includes detachably connecting a cosmetic container to a connecting portion of the applicator head and dispensing cosmetic fluid from the container through a fluid channel in fluid communication with a first side of the applicator head's housing. The cosmetic fluid is applied to the user's skin using at least one rolling element disposed on the first side of the housing. At the same time, at least one stimulation element on the first side is activated to emit light of a selected wavelength toward the skin. A display located on the second side of the housing presents treatment parameters or feedback, while a skin detection sensor on the second side detects at least one skin parameter. Based on the detected skin parameters, a control unit adjusts the operation of the stimulation element or the dispensing of the cosmetic fluid.

Several embodiments of the present invention will now be described in detail with references to FIGS.

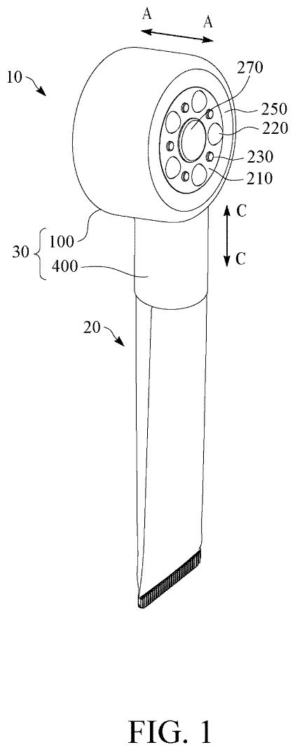

Referring to FIG. 1 to FIG. 4, an applicator head 10 comprising a main body 30 includes a housing 100 and a connecting portion 400 configured to detachably connect with a cosmetic container 20. A first side of the housing 100 has at least one stimulation element and at least one rolling element 220. A second side of the housing 300 includes a display 320 configured to provide visual information. A first fluid channel 411 is in fluid communication with the cosmetic container 20 and the first side of the housing 100. The first and second sides of the housing 100, 300 are arranged at an inclined orientation relative to a central axis of the main body 30 for ergonomic application.

The connecting portion 400 facilitates coupling of the housing 100 with the cosmetic container 20. The connecting portion 400 is provided with a first fluid channel 411, which is used to allow fluid in the cosmetic container 20 to flow to the first side of the housing 100.

In some embodiments, the first side 200 and the second side 300 of the housing 100 are arranged at an inclined orientation relative to a central axis of the main body 30. This allows the housing 100 to be appropriately enlarged, resulting in larger outer dimensions on two sides of the housing 100 spaced apart. This increases the space available for mounting the stimulation element within the housing 100. This, in turn, increases the contact area between the stimulation element and the user's skin, improving the delivery of the skincare fluid (which may be a serum, cream, Moisturizer, SPF lotion, SPF Moisturizer & Face Cream, or other skincare serum) to the user. The display 320 can be used to display the operating status of the applicator head 10 or other information to enrich the functionality of the applicator head 10.

In some embodiments, the first side 200 and the second side 300 of the housing 100 are inclined relative to one another so as to define an angle in the range of about 10 degrees to about 60 degrees with respect to a central axis of the main body 30. The inclined orientation enhances user comfort, precision of application, and visibility during use through an ergonomic design.

In this embodiment of the present application, when the connecting portion 400 is connected to the cosmetic container 20, the user can control the applicator head 10 by holding the cosmetic container 20 for skincare.

In another example, the second side of the housing 300 may include a lamp and a control switch. The control switch can be used to turn the lamp on or off, and can also be used to turn the first side of the housing 100 on or off. Exemplarily, the applicator head 10 also includes a circuit component 600. The lamp and the first side of the housing 100 are both electrically connected to the circuit component 600 via the control switch. Thus, by switching the control switch on and off, the lamp and the first side of the housing 100 can be simultaneously turned on or off.

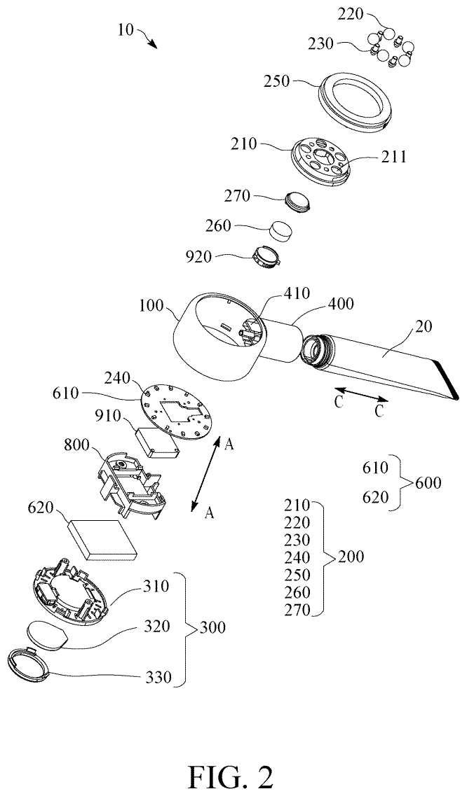

In this embodiment of the present application, as shown in FIG. 1 and FIG. 2, the connecting portion 400 is disposed on the outer circumference of the housing 100. By configuring the housing 100 as a cylindrical structure, the outer surface of the housing 100 is smoother, reducing angular features. This improves comfort during use and, to a certain extent, reduces the risk of injury from impacts, resulting in improved safety. However, in other embodiments, the housing may be non-cylindrical in shape.

Optionally, the connecting portion 400 comprises a threaded connector, bayonet mount, snap-fit mechanism, or magnetic locking mechanism to detachably connect with the cosmetic container 20. In one example, referring to FIG. 2 to FIG. 4, the connecting portion 400 is also a cylindrical structure.

The inner hole of the connecting portion 400 communicates with the inner hole of the housing 100. The cosmetic container 20 has a container mouth, which is inserted into the inner hole of the connecting portion 400. Typically, the container mouth of the cosmetic container 20 is connected to a container cap, and both the container mouth and the container cap are located in the inner hole of the connecting portion 400.

A pipe structure 410 is also provided in the inner hole of the connecting portion 400, forming a first fluid channel 411. The container cap is provided with a communication hole, which communicates with the fluid storage chamber of the cosmetic container 20 for storing the fluid.

One end of the pipe structure 410 is connected to the first side of the housing 100, and the other end is inserted into the communication hole, thereby connecting the first fluid channel 411 to the fluid storage chamber of the cosmetic container 20. This allows the fluid in the cosmetic container 20 to flow through the first fluid channel 411 to the first side of the housing 100.

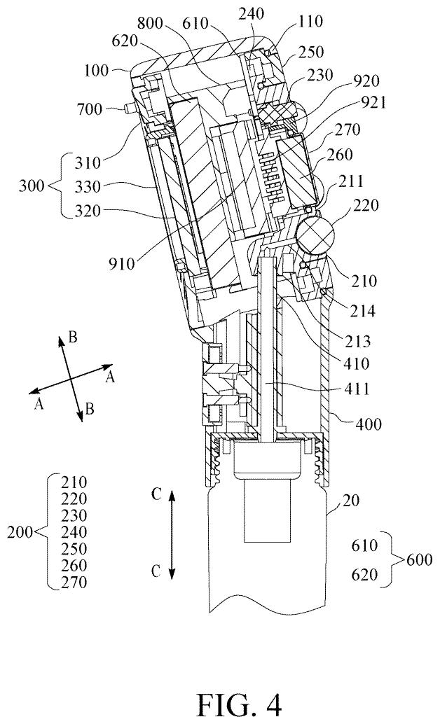

In this embodiment, as shown in FIG. 2 or FIG. 4, the applicator head 10 further includes a mounting bracket 800 and a circuit component 600. The mounting bracket 800 is mounted in the housing 100, and the circuit component 600 is mounted on the mounting bracket 800. The first side of the housing 100 and the second side of the housing 300 are respectively electrically connected to the circuit component 600, so that the circuit component 600 supplies power to the first side of the housing 100 and the second side of the housing 300, respectively.

In this embodiment of the present application, the circuit component 600 includes a circuit board 610 and a power supply battery. Both the circuit board 610 and the power supply battery are mounted on a mounting bracket 800. The power supply battery is electrically connected to the circuit board 610. The first side of the housing 100 and the second side of the housing 300 are both electrically connected to the power supply battery via the circuit board 610, so that the power supply battery supplies power to the first side 200 of the housing 100 and the second side 300 of the housing 100, respectively.

The mounting bracket 800 facilitates mounting the circuit component 600 within the housing 100. Specifically, when the housing 100 is cylindrical, the housing 100 has an inner hole, and the mounting bracket 800 is mounted within the inner hole of the housing 100.

In one embodiment, the first side of the housing 100 includes a mounting member 210 and the stimulation element. The mounting member 210 is located in an opening on the first side of the housing 100 and is connected to the housing 100. The stimulation element is mounted on a side of the mounting member 210 facing away from the second side of the housing 300. A raised portion 213 is provided on the side of the mounting member 210 facing the second side of the housing 300. A second fluid channel 214 is provided between the mounting member 210 and the raised portion 213. The second fluid channel 214 extends from the side of the raised portion 213 near the connecting portion 400 to the side of the mounting member 210 facing away from the second side of the housing 300. The first fluid channel 411 communicates with the second fluid channel 214.

During use, the user brings the applicator head 10 into contact with the skin by placing the side of the mounting member 210 facing away from the second side of the housing 300 in contact with the skin. With this arrangement, the fluid in the cosmetic container 20 can flow sequentially through the first fluid channel 411 and the second fluid channel 214 to the side of the mounting member 210 facing away from the second side of the housing 300, facilitating application of the fluid to the user's skin. Optionally, the end of the pipe structure 410 facing away from the cosmetic container 20 is inserted into the second fluid channel 214, thereby connecting the first fluid channel 411 with the second fluid channel 214.

Alternatively, the mounting member 210 and the housing 100 can be connected by any method, including snap-fitting, screwing, bonding, or welding, without limitation.

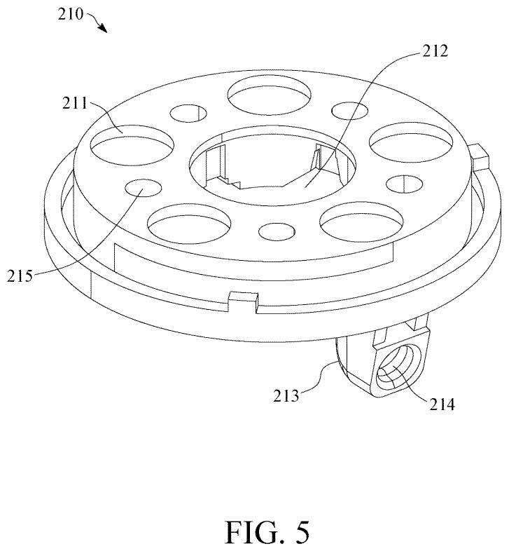

In yet other embodiments, referring to FIG. 1, FIG. 2, and FIG. 5, the first side of the housing 100 includes at least one rolling element 220. A mounting member 210 is provided with at least one receiving groove 211 on a side facing away from the second side of the housing 300. Each receiving groove 211 corresponds to each rolling element 220, and each rolling element 220 is movably mounted within the corresponding receiving groove 211. A second fluid channel 214 is connected to at least one receiving groove 211. That is, when the applicator head 10 includes only one rolling element 220, the number of receiving grooves 211 is also one, and the rolling element 220 is movably mounted in the receiving groove 211. The second fluid channel 214 is in communication with the receiving groove 211.

When the applicator head 10 includes multiple rolling elements 220, the number of receiving grooves 211 is equal to the number of rolling elements 220, and the multiple receiving grooves 211 are provided in a one-to-one correspondence with the multiple rolling elements 220. Each rolling element 220 is movably mounted in a corresponding receiving groove 211. The second fluid channel 214 may be in communication with one, some, or all of the receiving grooves 211. When the second fluid channel 214 is connected to the multiple receiving grooves 211, the second fluid channel 214 may specifically include a main channel and multiple sub-channels. The sub-channels are provided in a one-to-one correspondence with the multiple receiving grooves 211, with one end of each sub-channel connected to the corresponding receiving groove 211 and the other end connected to the main channel.

In this embodiment of the present application, part of the structure of the rolling element 220 is located in the receiving groove 211, while the other part of the structure extends out of the side of the mounting member 210 facing away from the second side of the housing 300.

During use, the user can place the rolling element 220 in contact with the skin and move the applicator head 10, causing the rolling element 220 to roll on the skin. The rolling process of the rolling element 220 evenly applies the fluid to the skin and massages the skin to promote skin absorption of the fluid.

In some embodiments, the applicator head 10 may include a vibrating massager that vibrates and massages the user's skin, also promoting skin absorption of the fluid.

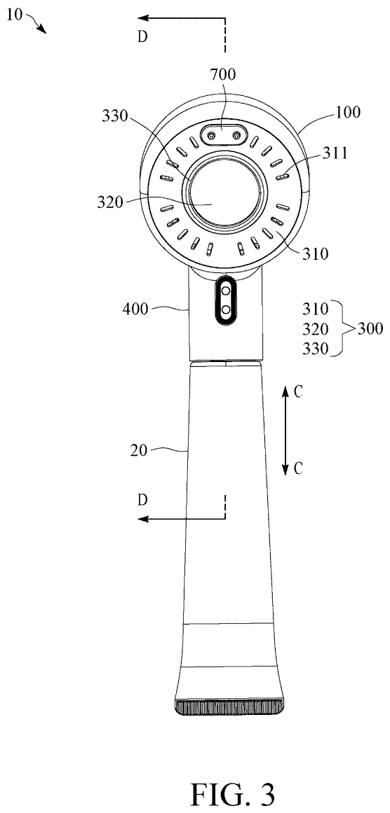

In one possible design, as shown in FIG. 3 and FIG. 4, the second side of the housing 300 includes a display screen 320 and a cover 310. The cover 310 covers the opening on the second side of the housing 100 and is connected to the housing 100. The cover 310 is formed with a mounting slot 313, with the opening of the mounting slot 313 facing away from the first side of the housing 100. The display screen 320 is mounted within the mounting slot 313. This facilitates mounting the display screen 320 in the opening on the second side of the housing 300 while also providing some protection for the display screen 320.

Specifically, the display screen 320 is electrically connected to the power supply battery in the circuit component 600 via the circuit board 610 in the circuit component 600. Alternatively, the cover 310 and the housing 100 may be connected by snapping, screwing, plugging, or any other method.

In some embodiments, the display screen 320 may include an SPF mirror that operates on the principle of ultraviolet (UV) light absorption by sunscreen formulations. When sunscreen is applied, the UV-blocking ingredients absorb or scatter the UV radiation, causing the covered areas of the skin to appear significantly darker in the mirror or on the display, while unprotected or unevenly covered areas appear lighter. This contrast enables users to clearly observe the distribution, thickness, and uniformity of sunscreen application, thereby highlighting gaps or insufficient coverage that are not visible under normal lighting conditions.

Optionally, when the applicator head 10 further includes a mounting bracket 800, the cover 310 may also be connected to the mounting bracket 800. Optionally, the cover 310 and the mounting bracket 800 may be connected using a connection structure such as screws or snaps.

In some embodiments, as shown in FIG. 2 to FIG. 4, the second side of the housing 300 further includes a retaining member 330. The retaining member 330 is located within the mounting slot 313 and is detachably connected to the cover 310. The retaining member 330 is located on the side of the display screen 320 facing the opening of the mounting slot 313. The retaining member 330 retains the display screen 320 within the mounting slot 313.

Optionally, the retaining member 330 may be a ring-shaped structure, a plate-shaped structure, or any other shape. In one example, the retaining member 330 is an annular structure.

At least a portion of the retaining member 330 is aligned with the outer edge of the display screen 320, enabling the retaining member 330 to retain the display screen 320 within the mounting slot 313 while facilitating viewing of the display screen 320 by the user. Furthermore, the retaining member 330 is made of a translucent material, such as acrylic or glass. The retaining member 330 has an annular inner cavity, within which multiple second light-emitting diodes are spaced apart.

In one embodiment, the second light-emitting diodes (LEDs) function as ultraviolet (UV) light sources and are utilized for detecting the sunscreen SPF (Sun Protection Factor) value. A display screen 320 is configured to operate as a sunscreen testing interface, presenting the measured or calculated SPF value, while a surrounding arrangement of the second LEDs (not shown) is disposed within a retaining member 330. The second LEDs are adapted to emit UV light onto a target surface, such as skin or a substrate coated with sunscreen, thereby facilitating visual or sensor-based evaluation of the sunscreen's protective efficacy against UV radiation. The centrally positioned display screen 320, in cooperation with the UV illumination, provides the SPF value in a clear and user-friendly manner, thereby delivering a compact and integrated system for real-time assessment of sunscreen performance.

In another example, the retaining member 330 is a plate-shaped structure. In this example, the retaining member 330 is also made of a light-transmitting material, such as acrylic or glass. The retaining member 330 covers the opening of the mounting slot 313. This ensures that the user's viewing of the display screen 320 through the retaining member 330 is not affected and provides improved protection for the display screen 320.

Optionally, the retaining member 330 and the cover 310 may be detachably connected by snapping, plugging, or other means.

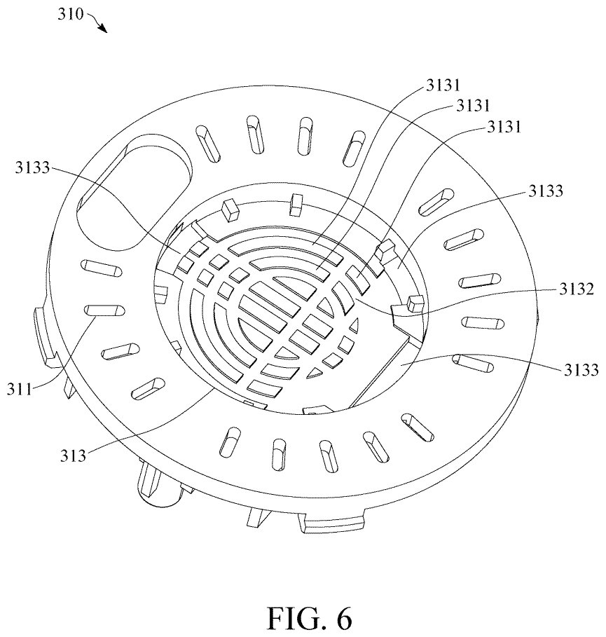

In some embodiments, as shown in FIG. 3 and FIG. 6, the cover 310 is provided with heat dissipation holes 311 extending through it. The heat dissipation holes 311 and the mounting slots 313 are spaced apart radially above the housing 100. The applicator head 10 also includes a heat dissipation fan 910, which is disposed within the inner aperture of the housing 100 and located between the first side of the housing 100 and the cover 310. The heat dissipation fan 910 is configured to drive air within the inner aperture of the housing 100 from the side proximal to the first side of the housing 100 to the side proximal to the cover 310, thereby dissipating heat within the housing 100 through the heat dissipation holes 311, and thereby achieving improved heat dissipation. Optionally, the heat dissipation fan 910 is mounted on the mounting bracket 800, or alternatively, the heat dissipation fan 910 may be connected to the inner wall of the housing 100. This is not intended to be a limitation.

Optionally, the cover 310 may include a flat plate structure and an extension structure. The flat plate structure covers the opening on the second side of the housing 300. The flat plate structure is connected to the housing 100 via a snap fastener. The extension structure is located on the side of the flat plate structure facing the first side of the housing 100. The mounting slot 313 extends from the side of the flat plate structure facing away from the first side of the housing 100 into the extension structure. This arrangement increases the depth of the mounting slot 313 by the extension structure, allowing the display screen 320 to be mounted within the mounting slot 313, resulting in a more compact structure and reducing the space occupied by the display screen 320 outside the housing 100.

Optionally, the heat dissipation holes 311 are specifically defined in the flat plate structure. Furthermore, there are multiple heat dissipation holes 311, which are spaced apart around the periphery of the extension structure.

In some embodiments, as shown in FIG. 6, the mounting slot 313 includes a bottom wall disposed opposite its opening. The bottom wall is provided with multiple ribs 3131. The bottom wall and any two adjacent ribs 3131 form a heat dissipation channel 3132. In this embodiment, the shapes of the ribs 3131 can be identical or different, and this is not a limitation. The provision of multiple ribs 3131 not only improves the structural strength of the cover 310 but also allows the display screen 320 to be spaced apart from the bottom wall. Any two adjacent ribs 3131 and the bottom wall form a heat dissipation channel 3132. Heat generated by the display screen 320 on the side facing the bottom wall of the mounting slot 313 is transferred through the heat dissipation channel 3132 to the gap between the display screen 320 and the side wall of the mounting slot 313, where it is dissipated through the gap between the display screen 320 and the side wall of the mounting slot 313.

In some embodiments, as shown in FIG. 4 and FIG. 6, the mounting slot 313 includes a bottom wall positioned opposite its opening and side walls surrounding the bottom wall. Ventilation holes 3133 are provided through at least one of the bottom wall and the side walls. The mounting slot 313 communicates with the inner hole of the housing 100 or the heat dissipation holes 311 through the ventilation holes 3133. This arrangement allows heat generated by the display screen 320 to be transferred to the inner hole of the housing 100 through the ventilation holes 3133.

The heat dissipation fan 910 then drives the air in the inner hole of the housing 100 toward the side near the cover 310, thereby quickly dissipating the heat generated by the display screen 320 to the outside of the housing 100 and improving heat dissipation.

In some embodiments, the mounting slot 313 includes a bottom wall positioned opposite its opening and side walls surrounding the bottom wall. The bottom wall is provided with multiple ribs 3131. The bottom wall and any two adjacent ribs 3131 form heat dissipation channels 3132. Ventilation holes 3133 are provided through at least one of the bottom wall and the side wall. The mounting slot 313 communicates with the inner hole or heat dissipation holes 311 of the housing 100 through the ventilation holes 3133. This arrangement allows heat generated by the display screen 320 on the side facing the bottom wall of the mounting slot 313 to be transferred to the inner hole of the housing 100 via the heat dissipation channels 3132 and the ventilation holes 3133. The heat is then driven by the heat dissipation fan 910 to flow air from the inner hole of the housing 100 toward the side near the cover 310, thereby quickly dissipating the heat generated by the display screen 320 to the outside of the housing 100, further enhancing heat dissipation. Optionally, the bottom wall or the side wall may be provided with ventilation holes 3133.

Alternatively, both the bottom wall and the side walls are provided with ventilation holes 3133. In one example, there may be multiple ventilation holes 3133, spaced apart, and each ventilation hole 3133 is formed at the junction of the bottom wall and the side walls.

In some embodiments, as shown in FIG. 4, a heat dissipation structure 920 is further provided within the inner hole of the housing 100. The heat dissipation structure 920 is provided with heat dissipation fins 921. The heat dissipation structure 920 is located between the heat dissipation fan 910 and the first side of the housing 100. The heat dissipation fins 921 are provided on the side of the heat dissipation structure 920 facing the heat dissipation fan 910.

The provision of the heat dissipation fins 921 allows heat generated by the first side of the housing 100 to be quickly transferred to the side of the heat dissipation structure 920 closer to the heat dissipation fan 910, thereby improving heat dissipation.

In some embodiments, as shown in FIG. 3 and FIG. 4, the applicator head 10 further includes a skin detection sensor 700 and a control unit. The skin detection sensor 700 is mounted on the housing 100, the second side of the housing 300, the first side of the housing 100, or the connecting portion 400. The control unit is mounted within the housing 100 and is signal-connected to the skin detection sensor 700 and the second side of the housing 300. The skin detection sensor 700 is configured to detect the skin condition/parameter and feed the detection results back to the control unit. The control unit is configured to control the second side of the housing 300 to display the detection results fed back by the skin detection sensor 700.

With this arrangement, the skin detection sensor 700 can detect the user's skin condition/parameter and display the detection results on the second side of the housing 300. This allows the user to understand their current skin condition through the second side of the housing 300, allowing them to select appropriate skincare products based on their skin condition. Optionally, the control unit specifically controls the display screen 320 of the second side of the housing 300 to display the detection results fed back by the skin detection sensor 700. Optionally, the skin detection sensor 700 is specifically used to detect the moisture and oil content of the skin.

In some embodiments, the skin detection sensor 700 can be mounted on any of the housing 100, the second side of the housing 300, the first side of the housing 100, or the connecting portion 400. For example, the skin detection sensor 700 can be mounted on the second side of the housing 300, specifically on the side of the cover 310 of the second side of the housing 300 facing away from the first side of the housing 100. During the skin condition/parameter detection process, the skin detection sensor 700 is directly brought into contact with the skin.

Alternatively, the skin detection sensor 700 can also be mounted on the outer peripheral surface of the housing 100.

In some embodiments, the applicator head 10 may also include a voice module, which is connected to the control unit via the circuit board 610. The voice module can produce sound, and the control unit is also used to control the voice module to announce the detection results of the skin detection sensor 700, the remaining power of the power supply unit 620, and the operating time of the first side of the housing 100.

In some embodiments, during the skin condition/parameter detection process, after the skin detection sensor 700 has been in contact with the skin for a predetermined time (e.g., 1 second, 3 seconds, or 4 seconds), the control unit may control the voice module to play the detection results of the skin detection sensor 700.

In one possible design, the first side of the housing 100 includes at least one positive electrode terminal and at least one negative electrode terminal, each of which is electrically connected to the circuit component 600. In this setup, the positive and negative electrode terminals are placed in contact with the user's skin, allowing microcurrent to flow through the user's skin. This micro-current stimulates the skin, enhancing skin cell activity and accelerating skin metabolism, helping to remove aging glial cells and leaving the skin smoother and more delicate.

In certain embodiments, the rolling element 220 may also facilitate this function. The lower surface of the rolling element 220 is coupled to the circuit component 600. When the rolling element 220 is formed of a metallic material, a micro-current is conducted from the circuit component 600 to the lower surface and subsequently through the metallic rolling element 220.

In some embodiments, there are two positive and two negative electrode terminals, each electrically connected to the circuit component 600. The first side of the housing 100 further includes the mounting member 210 and the rolling elements 220. The two positive and two negative electrode terminals are mounted on the mounting member 210 at intervals. As shown in FIG. 1 and FIG. 2, the rolling elements 220 include five balls. The mounting member 210 is provided with five receiving grooves 211, evenly spaced about the axis of the housing 100. Each ball is movably mounted in a corresponding receiving groove 211. An electrode terminal 230 is positioned between any two adjacent receiving grooves 211 in the circumferential direction around the housing 100.

In this embodiment, there are five electrode terminals 230. Two of these are connected to the positive electrode of the power supply unit 620 in the circuit component 600, while the other two are connected to the negative electrode of the power supply unit 620. The last electrode terminal 230 is not energized and serves a decorative purpose. It should be understood that the electrode terminal 230 connected to the positive electrode of the power supply unit 620 is a positive electrode terminal, while the electrode terminal 230 connected to the negative electrode of the power supply unit 620 is a negative electrode terminal.

Optionally, as shown in FIG. 4 and FIG. 5, the mounting member 210 is provided with through-holes 215. The number of through-holes 215 is equal to the number of electrode terminals 230, with five through-holes 215 corresponding to five electrode terminals 230. Each electrode terminal 230 is inserted into a corresponding through-hole 215, allowing the electrode terminal 230 to be electrically connected to the circuit board 610 while also being in contact with the user's skin.

In some embodiments, as shown in FIG. 2 and FIG. 4, the first side of the housing 100 includes at least one stimulation element. Stimulation elements are selected from the group consisting of a phototherapy component, electrotherapy component, magnotherapy component, micro-current component, heating element, cooling element, Peltier element, vibrational element for massage, ultrasonic wave component, or any other therapeutic component.

In this embodiment, the stimulation element is a phototherapy component. The phototherapy component comprises a light-emitting diode (LED) configured to emit light in a wavelength range selected from red, blue, green, purple, or near-infrared as required for targeted skincare treatments.

In this embodiment, the first light-emitting diode 240 is electrically connected to the circuit component 600 and can emit blue or red light. In this configuration, when the first light-emitting diode 240 emits blue light, it helps sterilize and remove acne, regulates sebaceous gland secretion, and strengthens the skin barrier. When the first light-emitting diode 240 emits red light, it promotes the skin's absorption of the fluid.

Alternatively, as shown in FIG. 2, there may be multiple first light-emitting diodes 240. These multiple first light-emitting diodes 240 are mounted on the circuit board 610 in the circuit component 600 at intervals around the axis of the housing 100. Each of the multiple first light-emitting diodes 240 is signal-connected to a control unit, whose light color is controlled by the control unit. Alternatively, the first light-emitting diode 240 may have the same structure as the second light-emitting diode, and further description is omitted.

The first side of the housing 100 also includes a lampshade 250, which is an annular structure and covers the side of the multiple first light-emitting diodes 240 facing away from the circuit board 610. The lampshade 250 is also made of a light-transmitting material, such as acrylic or glass.

The lampshade 250 is supported on the side of the circuit board 610 facing away from the second side of the housing 300. The lampshade 250 surrounds the outer periphery of the mounting member 210, with the outer edge of the lampshade 250 positioned between the circuit board 610 and the stopper 110. The stopper 110 and the mounting bracket 800 constrain the circuit board 610 and lampshade 250 to be stably positioned within the inner hole of the housing 100. The restraining action of the lampshade 250 and the circuit board 610 ensures that the mounting member 210 is stably mounted within the opening on the first side of the housing 100.

Optionally, as shown in FIG. 4, an annular stopper 110 protrudes from the inner wall of the opening of the housing 100. The axis of the stopper 110 coincides with the axis of the housing 100. The circuit board 610 is supported on the side of the mounting bracket 800 facing away from the second side of the housing 300.

In some embodiments, as shown in FIG. 2 or FIG. 4, the first side of the housing 100 includes a semiconductor cooling element 260, which is electrically connected to the circuit component 600 via the control unit. The control unit is configured to switch the direction of power flow to the semiconductor cooling element 260.

In some embodiments, the semiconductor cooling element 260 can be mounted on the mounting member 210 or the lampshade 250. The side of the semiconductor cooling element 260 facing away from the second side of the housing 300 is a working surface, which is intended to contact the user's skin. By switching the direction of power flow to the semiconductor cooling element 260 by the control unit, the working surface can be switched to either a cooling or heating surface, enabling the applicator head 10 to provide both cooling and heating effects. It is worth noting that the first side of the housing 100 may include only the semiconductor cooling element 260, which is directly connected to the housing 100.

In some embodiments, as shown in FIG. 2, FIG. 4, and FIG. 5, the mounting member 210 is provided with an avoidance hole 212 extending through it. The avoidance hole 212 is arranged to coincide with the axis of the housing 100. A protective cover 270 is also installed in the avoidance hole 212. The outer surface of the protective cover 270 is provided with a flange that contacts the side of the mounting member 210 facing the second side of the housing 300. The semiconductor cooling element 260 is located in the avoidance hole 212, and the protective cover 270 is positioned over the working surface of the semiconductor cooling element 260.

The protective cover 270 prevents the fluid from entering the inner hole of the housing 100 through the avoidance hole 212 and prevents the semiconductor cooling element 260 from directly contacting the user's skin, thereby improving the safety and reliability of the applicator head 10.

In one embodiment, the first side of the housing 100 includes the semiconductor cooling element 260 and the first light-emitting diode 240. Both the semiconductor cooling element 260 and the first light-emitting diode 240 are connected to the control unit signal via the circuit board 610. When the control unit controls the first light-emitting diode 240 to emit blue light, it can also control the semiconductor cooling element 260 to flow a forward current, switching the working surface of the semiconductor cooling element to the cooling surface. When the control unit controls the first light-emitting diode 240 to emit red light, it can also control the semiconductor cooling element 260 to flow a reverse current, switching the working surface of the semiconductor cooling element to the heating surface.

In one embodiment, the heat dissipation structure 920 is located between the semiconductor cooling element 260 and the heat dissipation fan 910. Multiple heat dissipation fins 921 are provided on the side of the heat dissipation structure 920 facing the heat dissipation fan 910. This arrangement facilitates rapid transfer of heat generated by the semiconductor cooling element 260 to the side of the heat dissipation structure 920 closest to the heat dissipation fan 910, thereby improving heat dissipation.

In one embodiment, the control unit is operatively connected to both the skin detection sensor 700 and the stimulation element. The control unit is configured to regulate the operation of the stimulation elements (e.g., light therapy, magneto therapy, vibration, micro-current, or ultrasonic stimulation) based on predefined treatment parameters, such as user-selected intensity levels or durations, and dynamically adjust output levels of the stimulation elements in response to the real-time skin parameters detected by the skin detection sensor 700. For instance, if the skin hydration is below a target threshold, the control unit may increase micro-current intensity or phototherapy duration.

The control unit may be preprogrammed with multiple treatment modes, including preset therapy cycles tailored for different skin types or conditions (e.g., oily, dry, sensitive, or combination skin). These treatment profiles allow for rapid user selection without manual calibration.

In further embodiments, the control unit is capable of storing treatment history and user-specific data in internal or cloud-connected memory. This data is used to generate customized therapy recommendations for future use, thereby personalizing the therapeutic experience.

The user interface in the applicator head 10 comprises one or more input components, such as physical buttons or a touchscreen display 320. The interface allows users to: select from available treatment modes; adjust therapy intensity levels, and activate or deactivate individual stimulation elements.

In a preferred embodiment, a user interface includes a touchscreen display 320 that provides real-time monitoring of both therapy parameters (such as duration, mode, and intensity) and skin parameter readings (such as hydration, SPF, or temperature levels). The display 320 also provides visual feedback on ongoing therapy sessions and progress.

The control unit may also incorporate a wireless communication module (e.g., Bluetooth or Wi-Fi), enabling the applicator head 10 to interface with a mobile application. The app can be used to: remotely control the applicator head; modify treatment settings; view stored data and skin analysis reports; and receive notifications and usage reminders. The mobile connectivity enhances convenience and expands the applicator head's usability for users who prefer remote management and data access.

In an embodiment of the present invention, the skin parameters detected by the skin detection sensors in the applicator head 10 can be transferred to a mobile device using an e-communication channel, such as NFC, IR, BLE, or Wi-Fi. When the applicator head 10 is brought close to the mobile device, the communication channel transfers the data detected by the applicator head 10 to the mobile phone and displays it on the mobile phone. The mobile phone is equipped with a mobile application corresponding to the applicator head 10, and the mobile application is used to display the skin condition/parameter detected by the skin detection sensor 700.

To prevent overuse and ensure safety, the applicator head 10 is configured with an automatic shut-off function. This feature automatically terminates the operation of the stimulation elements after a predefined therapy duration, based on system defaults or user settings.

This intelligent and ergonomic applicator head 10, integrating real-time skin sensing, adaptive control logic, user feedback, and mobile connectivity, provides a comprehensive and customized therapeutic experience, improving both usability and treatment outcomes for users across diverse skin profiles.

The above description is merely an optional embodiment of the present application and is not intended to limit the present application. Any modifications, equivalent substitutions, and improvements made within the spirit and principles of the present application shall be included within the scope of protection of the present application.

1. An applicator head comprising:
a main body includes a housing, a connecting portion coupled to the housing, the connecting portion is configured to detachably connect with a cosmetic container;
a first side of the housing includes at least one phototherapy element and at least one rolling element;
a second side of the housing includes a display configured to provide visual information;
a fluid channel in fluid communication with the cosmetic container and the first side of the housing;
wherein the first side of the housing and the second side of the housing are positioned at an inclined orientation relative to a central axis of the main body.

2. The applicator head of claim 1, wherein the at least one phototherapy element comprises a light-emitting diode (LED) configured to emit light in a wavelength range selected from red, blue, green, purple, or near-infrared as required for targeted skincare treatments.

3. The applicator head of claim 1, wherein the at least one rolling element is configured to rotate freely to provide a massage effect during application of the cosmetic fluid to the skin.

4. The applicator head of claim 1, wherein the at least one rolling element is made of a material selected from stainless steel, Jade, ceramic, or a thermally conductive polymer to provide cooling or heating during application.

5. The applicator head of claim 1, wherein the display is a touchscreen configured to provide real-time monitoring of both therapy parameters (such as duration, mode, and intensity) and skin parameter readings (such as hydration, SPF, or temperature levels).

6. The applicator head of claim 1, wherein the inclined orientation between the first and second sides of the housing forms an angle between 10 degrees and 60 degrees relative to the central axis of the main body.

7. The applicator head of claim 1, wherein the connecting portion comprises a threaded connector, bayonet mount, snap-fit mechanism, or magnetic locking mechanism to detachably connect with the cosmetic container.

8. An applicator head comprising:
a main body includes a housing, a connecting portion coupled to the housing, the connecting portion is configured to detachably connect with a cosmetic container;
a first side of the housing includes at least one stimulation element;
a second side of the housing includes at least one skin detection sensor to sense at least one skin parameter and a display configured to provide visual information;
a fluid channel in fluid communication with the cosmetic container and the first side of the housing to dispense a cosmetic fluid to a user's skin;
a control unit configured to:
coordinate dispensing of the cosmetic fluid with operation of the at least one stimulation element; and
adjust operation based on the detected at least one skin parameter and provide corresponding feedback via a user interface;
wherein the first side of the housing and the second side of the housing are positioned at an inclined orientation relative to a central axis of the main body.

9. The applicator head of claim 8, wherein the at least one stimulation element is selected from the group consisting of a phototherapy component, electrotherapy component, magneto therapy component, micro-current component, heating element, cooling element, Peltier element, vibrational element for massage, ultrasonic wave component, or any other therapeutic component.

10. The applicator head of claim 8, wherein the at least one skin detection sensor is selected from the group consisting of a temperature sensor, a moisture sensor, an impedance sensor, an optical sensor, a SPF sensor/UV camera, or a combination thereof.

11. The applicator head of claim 8, wherein the skin detection sensor is configured to detect at least one of a water level, oil level, temperature, SPF, or hydration level of the skin.

12. The applicator head of claim 8, wherein the control unit includes a wireless communication module for connectivity for connection to a mobile device to transmit skin data and receive treatment settings.

13. The applicator head of claim 8, the first side of the housing, and the second side of the housing are electrically connected to a circuit component.

14. The applicator head of claim 13, the first side of the housing includes at least one positive electrode terminal and at least one negative electrode terminal, each of which is electrically connected to the circuit component.

15. The applicator head of claim 8, wherein the display provides real-time visual feedback on the skin parameters and suggested treatment adjustments.

16. The applicator head of claim 8, wherein the control unit is programmed with multiple treatment modes, including pre-set therapy cycles based on skin conditions/parameters.

17. The applicator head of claim 8, wherein the control unit is configured to store user treatment history and provide customized therapy recommendations.

18. The applicator head of claim 9, further comprising a rechargeable battery or power source configured to supply power to the phototherapy element and display.

19. A method for delivering a cosmetic treatment to a user's skin using an applicator head, the method comprising:
detachably connecting a cosmetic container to a connecting portion of an applicator head;
dispensing a cosmetic fluid from the cosmetic container through a fluid channel in fluid communication with a first side of a housing of the applicator head;
applying the cosmetic fluid to the user's skin using at least one rolling element disposed on the first side of the housing;
activating at least one stimulation element on the first side of the housing to emit light of a selected wavelength toward the user's skin;
displaying skin condition/parameter and treatment parameters or feedback on a display located on a second side of the housing;
detecting at least one skin parameter using a skin detection sensor disposed on the second side of the housing; and
adjusting the operation of the stimulation element or dispensing of the cosmetic fluid based on the detected skin parameters via a control unit.

20. The method of claim 19, wherein the control unit adjusts the intensity or duration of the stimulation element based on real-time skin parameter readings.
